SEQUENCE LISTING (5 sequences)

SEQ ID NO: 1 - DNA, 4592 bp, Danio rerio
aagagttctagattatcttcttttgttttgcttggccgtccttgaacgagactgaaaaccagaataagagggctcttgaatctgtccacgcgtgaaaaatgcccgtgcgccgcggacatgtcgctctccagaacacctatctggacaccatcattaggaaattcgacgggcaaaatcgtaaattcttgatcgccaacgcccagatgaagaactgtggaatcatttactgcaatgagggcttttgtcagatgtttggcttctcgcgggcggagatcatgcagcagtcctgcacgtgtcagtttctggtggggccgggcactatgaagagcgccctgggacagctggcacaggctctgctcgggtcagaggagcgcaaagtggagatcctctactactctaaagaagggacttgtaggccgtgtttgattgatgtcattccggtaaagaatgaggaaggagttgtgatcatgttcatcctaaacttccaggaacttttagacccatccatgaagaaaggtggtctgaaacagcgcatggcgaacagctggctgcgggcaggtcagcgacgcaggatgcatctgagaatgccttctctccgtgtcaaaaggcagccgtctcttcctaaagaccactttgagggagtagtggtggattacctacagccgtctcatgaggaggtggccctcaaagatctccagatgtctccagacagctgtttaaagtcagagacgcaggccttgattcagcagaccccatcctcctgcgaactctcaccgccaccctcacggccctcagaccgactggagccaagtggaccccttctcaaacatagccactccagagagagcatgcacagcctgcgccgggcatcctcgctgcacgacatcgacggcatgagggaccagtggagcgatctaaagcccagtaatttgaactccacatcagactctgatttgatgcggcaccgcacaattggccgcatccctcaggtgacgatatccttcggctcggaccgcctcagaccaccttcccctactgagatcgaaatcatcgcacccagcaagattaaagaccgctcacagaacgtctcagaaaaggtcactcaagttacgcaggtcctgtcactcggggcagatgtgcttccggagtacaagcttcaagctccccgcatccacaaatggacaatcctgcactacagcccgttcaaggcggtgtgggactggatcatcctgctgctggtgctctacacggccgtcttcacgccatattcagccgcgttcctcctcaacgagcaggaggatgagagaagacgcacctgtggctacacctgcaaccctctgaacgtggtggacctggtggtggacgtcatgttcatcatcgacatcctcatcaacttcaggaccacatacgtcaatcacaacgacgaggtggtcagcaaccctgcccgcatcgctcagcactacttcaagggctggttcctcatagacatagttgctgccatccccttcgacctgctcattttcagatccggttcagatgagcctcaaaccaccactctgattggtctgctgaagactgcacggctgctgcgactggtgcgagtagcgagaaaactggaccgttactctgagtatggagcggcggtgctcttcctcctcatgtgcacctttgctctcatcgcccactggctggcatgcatctggtacgccattggcaatatggagcgcaccagctctgcccgcataggaggcatgaagatcggctggctggacaacttagctgaccaaattggcaagcagtacaatgacagcaacagtttctctggcccctccatcaaggacaaatatgttactgcattgtacttcaccttcagtagcttgaccagtgtgggcttcggaaatgtctctccaaacaccaaccctgagaagattttctccatttgcgtcatgcttattggctctctgatgtatgccagtatctttggcaatgtgtctgctatcattcagaggttgtattctggcacagcacggtaccacactcagatgctccgtgtgaaagagttcatcagatttcatcagatcccaggtggccttcggcaaagactagaggagtatttccagcatgcctggccctacaccaacggcatcgacatgaacgctgtgctgaagggttttccagagtgtctacaggctgacatctgtcttcatctgaaccgcagtctgcttcaaagctgtaaggcatttcgaggggcaagtaagggctgtctacgggctctggccatgcgctttaggaccactcatgcccctcccggcgacactctggttcacagtggagacgtactgaccgcactctacttcatctcccgtggctccatagagatcctcagagacgatgtggtcgtggccattttaggtaagaatgacattttcggggagcccatcagtctatatgccaggccggggaagtcaagcgctgatgtgagggctctgacatactgtgacctgcataagatcctgagggacgacctgctggaggtgctggacatgtaccccgatttctccgacaacttctggagcaacttggagatcacsttcaaccttcgagatgtagacagaatcatgcacccaacaccaagcgaggactcagattgtgggtacaggcggcccagacaccggaggaaccccctacggcgaaatcgacctgatgggatggaccgggatgggatggacacatacccagtccagccctgttctcctgtggggaaccaccggggggcgataccgctgtcacagtgggatgagttgtgtagtgatggcagccctgcttctctctccagtgaagaggatatgaagcctctggtgtccggccagggggatatgtactcactgggcacagagatgcaggaattctccccttctgcagtcagtcttatgccctccgcccacagcactgcttctgccatggctggaccgctgacaggtgcacatcagtacacagcagcccctctgaacatttcaggggtatacagctatttgtcggaccgcagggccagtgagtactcagagagtcagagacgttcctctgcggtgcaggcatgctatcaccatcacagcccctgtgtgggggacagaccgaatcaactacaggcccgtctggagctgctgcagtcacaactcaacagactggagaccaggatgacggctgatataaatgtgatcctgcagctgctgcagaggcagatggcacctgttcctccagcctacagtgccgtttctcctgatccactcgcacacccagtccccccagcccaccccaccagcctctacactacagcagcacacaacaccacaccctccctacagatcactgacgcttcttcacctggaaagagtcctgacgtggacagcttgaaagagaagtctccagactctctgtccagcgggattcatctgacggtggcttccactgacaccatgtccatgtctccagagacagagctgtctgtgccctctgctgggcctcttctccagcctccaggcctgctgtgcagtagcctgcgcttcccgtcactcccagacagcctggagggtcctggcacactggaggggtcacccgaaatccaaaggcacgtgtccgatccagtcttacccggaagctagaggctaaaaagcagactattgttcattctgagactcatttactccacggtttccaatattgcacttttgcaacaaaatgtattcattaggacgtccgttagagcaaacgagccgatttctttgaatctctgaggtttgtctcatatcccttctcactaagagcaggaagttctcttctgtcgcgacagtatttggtaacttttgtttgcaaccgaatatgacaaaaagcgcagtgggcaataaagacgagatccaagagtcaaagagagatgccacattacagttcacccgcctccatcttgtttgtttgtgtctagtttgaaattgtcctcctagtctaaacctcatttttttgtctgcttttgtcaaactaagattcacaggacagattcactaaagtagaacaaaaaaatgttcaaagtttttcttttcagtgtgtacattttctaattctatatattttgtgtgtggttagctgtttgacccaaatctaatgtgttactgcatcagctttatgactggcaccaaatgaatctgtgtgtttggattcattgtaaccaaatgtgcttgaattcagggttatgaaatgattttcctcactgaagtgtgactgactgagtgtctgtgtgtgtgtgtgtgagtgtagatttatgatgtgtgatctttttggaaatgtgattttgaatatgataaagaaaaacattcaggcctaaaggatttggagttctcaaggttttggatggaagaaagtgaaggactgtttttgtaaatcactgttgagatgaaatcagtgtaaagatcatttcacctctgaaatagaagcagatgaatgtaaaatgattggaagcctttattggactgtaaaggtttccaaatgcacacaactgttattattataattatttgtttagattaaatgttatttataaaaaa

SEQ ID NO: 2 - protein, 1186 aa, Danio rerio
MPVRRGHVALQNTYLDTIIRKFDGQNRKFLIANAQMKNCGIIYCNEGFCQMFGFSRAEIMQQSCTCQFLVGPGTMKSALGQLAQALLGSEERKVEILYYSKEGTCRPCLIDVIPVKNEEGVVIMFILNFQELLDPSMKKGGLKQRMANSWLRAGQRRRMHLRMPSLRVKRQPSLPKDHFEGVVVDYLQPSHEEVALKDLQMSPDSCLKSETQALIQQTPSSCELSPPPSRPSDRLEPSGPLLKHSHSRESMHSLRRASSLHDIDGMRDQWSDLKPSNLNSTSDSDLMRHRTIGRIPQVTISFGSDRLRPPSPTEIEIIAPSKIKDRSQNVSEKVTQVTQVLSLGADVLPEYKLQAPRIHKWTILHYSPFKAVWDWIILLLVLYTAVFTPYSAAFLLNEQEDERRRTCGYTCNPLNVVDLVVDVMFIIDILINFRTTYVNHNDEVVSNPARIAQHYFKGWFLIDIVAAIPFDLLIFRSGSDEPQTTTLIGLLKTARLLRLVRVARKLDRYSEYGAAVLFLLMCTFALIAHWLACIWYAIGNMERTSSARIGGMKIGWLDNLADQIGKQYNDSNSFSGPSIKDKYVTALYFTFSSLTSVGFGNVSPNTNPEKIFSICVMLIGSLMYASIFGNVSAIIQRLYSGTARYHTQMLRVKEFIRFHQIPGGLRQRLEEYFQHAWPYTNGIDMNAVLKGFPECLQADICLHLNRSLLQSCKAFRGASKGCLRALAMRFRTTHAPPGDTLVHSGDVLTALYFISRGSIEILRDDVVVAILGKNDIFGEPISLYARPGKSSADVRALTYCDLHKILRDDLLEVLDMYPDFSDNFWSNLEITFNLRDVDRIMHPTPSEDSDCGYRRPRHRRNPLRRNRPDGMDRDGMDTYPVQPCSPVGNHRGAIPLSQWDELCSDGSPASLSSEEDMKPLVSGQGDMYSLGTEMQEFSPSAVSLMPSAHSTASAMAGPLTGAHQYTAAPLNISGVYSYLSDRRASEYSESQRRSSAVQACYHHHSPCVGDRPNQLQARLELLQSQLNRLETRMTADINVILQLLQRQMAPVPPAYSAVSPDPLAHPVPPAHPTSLYTTAAHNTTPSLQITDASSPGKSPDVDSLKEKSPDSLSSGIHLTVASTDTMSMSPETELSVPSAGPLLQPPGLLCSSLRFPSLPDSLEGPGTLEGSPEIQRHVSDPVLPGS

SEQ ID NO: 3 - DNA, 24 bp, Danio rerio
gacatgtccgcggcgcacgggcat

SEQ ID NO: 4 - protein, 1177 aa, Artificial Sequence
MPVRRGHVALQNTYLDTIIRKFDGQNRKFLIANAQMKNCGIIYCNEGFCQMFGFSRAEIMQQSCTCQFLVGPGTMKSALGQLAQALLGSEERKVEILYYSKEGTCRPCLIDVIPVKNEEGVVIMFILNFQELLDPSMKKGGLKQRMANSWLRAGQRRRMHLRMPSLRVKRQPSLPKDHFEGVVVDYLQPSHEEVALKDLQMSPDSCLKSETQALIQQTPSSCELSPPPSRPSDRLEPSGPLLKHSHSRESMHSLRRASSLHDIDGMRDQWSDLKPSNLNSTSDSDLMRHRTIGRIPQVTISFGSDRLRPPSPTEIEIIAPSKIKDRSQNVSEKVTQVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVKEFIRFHQIPGGLRQRLEEYFQHAWPYTNGIDMNAVLKGFPECLQADICLHLNRSLLQSCKAFRGASKGCLRALAMRFRTTHAPPGDTLVHSGDVLTALYFISRGSIEILRDDVVVAILGKNDIFGEPISLYARPGKSSADVRALTYCDLHKILRDDLLEVLDMYPDFSDNFWSNLEITFNLRDVDRIMHPTPSEDSDCGYRRPRHRRNPLRRNRPDGMDRDGMDTYPVQPCSPVGNHRGAIPLSQWDELCSDGSPASLSSEEDMKPLVSGQGDMYSLGTEMQEFSPSAVSLMPSAHSTASAMAGPLTGAHQYTAAPLNISGVYSYLSDRRASEYSESQRRSSAVQACYHHHSPCVGDRPNQLQARLELLQSQLNRLETRMTADINVILQLLQRQMAPVPPAYSAVSPDPLAHPVPPAHPTSLYTTAAHNTTPSLQITDASSPGKSPDVDSLKEKSPDSLSSGIHLTVASTDTMSMSPETELSVPSAGPLLQPPGLLCSSLRFPSLPDSLEGPGTLEGSPEIQRHVSDPVLPGS

SEQ ID NO: 5 - protein, 1159 aa, Homo sapiens
MPVRRGHVAPQNTFLDTIIRKFEGQSRKFIIANARVENCAVIYCNDGFCELCGYSRAEVMQRPCTCDFLHGPRTQRRAAAQIAQALLGAEERKVEIAFYRKDGSCFLCLVDVVPVKNEDGAVIMFILNFEVVMEKDMVGSPAHDTNHRGPPTSWLAPGRAKTFRLKLPALLALTARESSVRSGGAGGAGAPGAVVVDVDLTPAAPSSESLALDEVTAMDNHVAGLGPAEERRALVGPGSPPRSAPGQLPSPRAHSLNPDASGSSCSLARTRSRESCASVRRASSADDIEAMRAGVLPPPPRHASTGAMHPLRSGLLNSTSDSDLVRYRTISKIPQITLNFVDLKGDPFLASPTSDREIIAPKIKERTHNVTEKVTQVLSLGADVLPEYKLQAPRIHRWTILHYSPFKAVWDWLILLLVIYTAVFTPYSAAFLLKETEEGPPATECGYACQPLAVVDLIVDIMFIVDILINFRTTYVNANEEVVSHPGRIAVHYFKGWFLIDMVAAIPFDLLIFGSGSEELIGLLKTARLLRLVRVARKLDRYSEYGAAVLFLLMCTFALIAHWLACIWYAIGNMEQPHMDSRIGWLHNLGDQIGKPYNSSGLGGPSIKDKYVTALYFTFSSLTSVGFGNVSPNTNSEKIFSICVMLIGSLMYASIFGNVSAIIQRLYSGTARYHTQMLRVREFIRFHQIPNPLRQRLEEYFQHAWSYTNGIDMNAVLKGFPECLQADICLHLNRSLLQHCKPFRGATKGCLRALAMKFKTTHAPPGDTLVHAGDLLTALYFISRGSIEILRGDVVVAILGKNDIFGEPLNLYARPGKSNGDVRALTYCDLHKIHRDDLLEVLDMYPEFSDHFWSSLEITFNLRDTNMIPGSPGSTELEGGFSRQRKRKLSFRRRTDKDTEQPGEVSALGPGRAGAGPSSRGRPGGPWGESPSSGPSSPESSEDEGPGRSSSPLRLVPFSSPRPPGEPPGGEPLMEDCEKSSDTCNPLSGAFSGVSNIFSFWGDSRGRQYQELPRCPAPTPSLLNIPLSSPGRRPRGDVESRLDALQRQLNRLETRLSADMATVLQLLQRQMTLVPPAYSAVTTPGPGPTSTSPLLPVSPLPTLTLDSLSQVSQFMACEELPPGAPELPQEGPTRRLSLPGQLGALTSQPLHRHGSDPGS

(19) United States
(12) Patent Application Publication
(10) Pub. No.: US 2003/0074682 A1
(43) Pub. Date: Apr. 17, 2003

(54) Isolation, characterization, and use of a novel teleost potassium channel

(76) Inventor: Ulrike Langheinrich, Tubingen (DE)

Correspondence Address: JAN P. BRUNELLE, EXELIXIS, INC., 170 HARBOR WAY, P.O. BOX 511, SOUTH SAN FRANCISCO, CA, 94083-0511, US

(21) Appl. No.: 10/193,692

(22) Filed: Jul. 11, 2002

Related U.S. Application Data
(60) Provisional application No. 60/305,396, filed on Jul. 13, 2001.

(51) Int. Cl. A01K 67/027; C12Q 1/68; C07H 21/04

(52) U.S. Cl. 800/20; 514/44; 435/6; 536/23.5

(57) ABSTRACT

The present invention provides nucleic acid and polypeptide sequences associated with teleost ERG genes, which encode ERG family potassium channels. The invention further provides teleost models for cardiac function and methods of screening for cardio-active agents using mutant teleost larvae having reduced teleost ERG activity.

### REFERENCE TO RELATED APPLICATIONS

This application claims priority to the U.S. provisional patent application serial no. 60/305,396 filed Jul. 13, 2001. The content of the prior application is hereby incorporated in its entirety.

### BACKGROUND

Long QT syndrome (LQT) is an abnormality of cardiac muscle repolarization that predisposes affected individuals to lethal arrhythrmias and may be acquired (i.e., drug-induced) or congenital.

The administration of a wide range of drugs to predisposed patients is associated with completely undesired side effects, including life-threatening ventricular tachycardia, called torsade-de-pointes. These compounds belong to different pharmacological classes (antiarrhythmics, antidepressants, antifungals, antihistamines, neuroleptics, prokinetic drugs, antimicrobials) and prolong the ventricular repolarization and thus the QT interval of the electrocardiogram (Taglialatela et al., 1999; Crumb and Cavero, 1999; Taglialatela et al., 2000; Yap and Camm, 1999). Due to several cases of sudden death resulting from such arrhythmia, the drugs astemizole, terfenadine and cisapride have been withdrawn from the US market.

Most repolarization-lengthening drugs (e.g., cisapride [Rampe et al., 1997], astemizol [Suessbrich et al., 1996], terfenadine [Suessbrich et al., 1996], sotalol [Numaguchi et al., 2000], sertindole [Rampe et al., 1998], pimozide [Kang et al., 2000], sildenafil [Geelen et al., 2000], haloperidol [Suessbrich et al., 1997] and thioridazine [Drolet, 1999]) block the rapidly activating delayed rectifier current K⁺ current I_{Kr}, as demonstrated in electrophysiological studies with cardiac myocytes. The human ether-a-go-go-related gene (HERG) is believed to encode the protein that co-assembles with the small protein KCNE2 (MIRPI), to form I_{Kr}, (Sanguinetti et al., 1995; Abbott et al., 1999).

Inward rectifiers are a large class of potassium channels that preferentially conduct inward potassium currents at voltages negative to the potassium equilibrium potential. In the heart, these channels also have small outward conductances that regulate the resting potential and contribute to the terminal phase of repolarization (phase 3). At positive voltages, these channels close and thus help maintain the level of the resting potential. A structural explanation for how so many commonly used medications block HERG has been proposed, showing that cisapride, astemizole and terfenadine interact with two amino acids in the S6 domain of the channel (Mitcheson et al., 2000).

More than 90 HERG loss-of-function mutations that lead to the hereditary Long QT syndrome, LQTS2, are known. Like the acquired LQT, the congenital LQTS2 is also associated with syncope and sudden death due to repolarization abnormalities and the onset of rare but life-threatening torsades de pointes (Itoh et al. 1998; Splawski et al., 2000; January et al., 2000; Vatta et al., 2000).

While no animal model of LQTS2 exists, there are some animal models for LQT. A transgenic mouse model has been developed for LQTS1, a related disease caused by mutations in the KCNQ1 gene (Demolombe et al., 2001). In another model, overexpression of a mutated form of murine HERG (MERG) in mice leads to the expected QT-prolongation on a single cell level, but not in the intact animal (Babij et al., 1998), possibly due to the occurrence of several MERG isoforms.

Currently, candidate drugs are tested for putative QT-prolongation either in vitro or in vivo before their introduction on the market. Exemplary in vitro techniques use electrophysiological studies on HERG channels expressed in oocytes or ECG measurements on isolated purkinje fibers. For in vivo studies, laboratory animals, preferably anaesthetized dogs or rabbits, are injected with the drugs, and the QT-interval of the ECG is subsequently determined. These methods are associated with various limitations. Deficiencies in in vitro testing may include artifacts due to preparation and storage of cells, cell-to-cell variability, and low throughput. Deficiencies in in vivo testing may include species variability in sensitivity towards drugs, generally small effects as compared to standard deviation, dependency of effects of on pharmacokinetics, low throughput and high costs.

Thus, animal models of LQTS2 could be of great value to medical and pharmacological research, particularly for developing anti-arrhythmic agents to treat LQT, and for further studies of HERG-related atrio-ventricular block.

Zebrafish and other teleost fish provide effective animal models for mammals and humans; such models are useful for studying particular pathologies, as well as agents that promote or ameliorate such pathologies. For instance, PCT patent application WO9942606 discloses a method for screening agents for angiogenesis or cell death activity, and U.S. Pat. No. 5,565,187 discloses a method for studying capillary circulation in teleost. The zebrafish, Danio rerio, a cyprinid teleost fish, is becoming a leading vertebrate model organisms due to the relative ease of breeding (high number of progeny and short generation time), rapid development, and transparency during the first week of development. The heart, which lies just beneath the skin, can be easily studied by visual inspection of anaesthetized larvae with a stereo microscope. Furthermore, the early onset of a regular heartbeat at 30 hours post-fertilization allows detailed observation of cardiac function at early stages of development. Studies addressing the effect of small molecule compounds on zebrafish heart have been reported (e.g., Peterson et al., 2000). Zebrafish larvae are permeable to small molecules, and, due to the prominent location of the heart just beneath the skin, agents acting on the heart rapidly reach their target.

Medaka (Oryzias latipes) is also being developed as a model genetic organism. Like zebrafish, medaka has the advantages of ease of breeding and transparent embryos. Moreover, a large number of genetic and genomic tools have become or are becoming available for medaka. These include inbred strains, a genome-wide likage map, mutagenesis protocols, transgenic techniques, antisense knockdown techniques, and EST and genomic sequence, among others (see, e.g., Wittbrodt et al., 2002).

### SUMMARY OF THE INVENTION

The invention provides novel polynucleotide and polypeptide sequences associated with teleost ERG genes, which encode ERG family potassium channels. An exemplary teleost ERG is zebrafish ZERG, whose disruption in zebrafish larvae is associated with an abnormal heart beat phenotype.

In one aspect, the invention provides an isolated teleost ERG nucleic acid molecule that hybridizes under high stringency conditions to a nucleic acid molecule having the nucleic acid sequence presented as SEQ ID NO:1, or the complement thereof. In other embodiments, the teleost ERG nucleic acid molecule encodes the ZERG polypeptide having the amino acid sequence presented as SEQ ID NO:2 or comprises the polynucleotide sequence presented as SEQ ID NO:1 or the complement thereof.

The invention provides antisense oligomers capable of inactivating teleost ERG genes. Preferred antisense oligomers are capable of inactivating ZERG and comprise a nucleotide sequence complementary to at least 10 contiguous nucleotides within nucleotides 1-150 of SEQ ID NO:1, and preferably comprise a nucleotide sequence complementary to 20-30 contiguous nucleotides within nucleotides 1-130 of SEQ ID NO:1. Further preferred antisense oligomers are PMOs; an exemplary PMO of the invention has the nucleotide sequence presented as SEQ ID NO:3. The invention further provides genetically modified teleost in which the teleost ERG gene has been specifically disrupted by administration of an antisense oligomer of the invention.

The invention provides methods for screening for cardio-active agents using mutant teleost larvae having reduced teleost ERG activity. Candidate cardio-active agents are identified by their ability to modify the cardiac phenotype of such mutant teleost larvae. Preferred cardiac phenotypes include irregular arrhythmia, bradycardia, 2:1 arrhythmia, rescue of 2:1 arrhythmia, aberrant heart morphology, lack of circulation, and blood accumulation in the yolk. Mutant teleost larvae include zebrafish larvae having a mutation in an endogenous ZERG gene, and wild-type zebrafish larvae treated with ZERG-specific PMO oligonucleotides. Methods of this invention may be used to identify both pro-arrhythmic and anti-arrhythmic agents.

The invention provides chimeric teleost ERG genes encoding chimeric polypeptides that comprise sequences from both the teleost ERG polypeptide and the HERG polypeptide. Exemplary chimeric ZERG genes, such as the chimeric ZERG gene encoding the polypeptide having the sequence presented as SEQ ID NO:4, are provided. The invention further provides transgenic teleost comprising a chimeric gene of the invention.

### BRIEF DESCRIPTION OF THE FIGURE

FIG. 1 depicts a sequence alignment of human HERG and zebrafish ZERG protein sequences. The alignment was generated from a Clustal W multisequence alignment that also included ERG sequences from rabbit, dog, and mouse (ERG 1a). Specific domains are indicated above corresponding sequence and are described in detail herein.

### DETAILED DESCRIPTION OF THE INVENTION

The present invention provides a novel teleost potassium channel that is useful for the study of cardiac function. The present invention further provides methods for studying QT-prolongation using teleosts. As used herein, the terms “fertilized teleost alevin” and “teleost larvae” refer to fertilized teleost eggs. “Teleosts” include zebrafish (Danio rerio) and medaka (Oryzias latipes).

The present invention concerns the identification and characterization of ZERG, a novel zebrafish ortholog of the HERG gene (nucleotide, Genbank Identifier [GI] 4557728; protein, GI 4557729). ZERG nucleic acid and protein sequences are provided in SEQ ID NO:1 and SEQ ID NO:2, respectively. The ZERG mutant phenotype was previously characterized as the breakdance (bre, tb218) mutant. The bre phenotype was identified during a large-scale zebrafish screen for early developmental defects (Chen et al.) but was not previously linked to a specific mutant gene. Homozygous breakdance larvae display an abnormal heartbeat, specifically, a 2:1 beat ratio such that the ventricle contract once while the atrium contracts twice. This abnormal heartbeat is hereinafter also referred to as “2:1 arrhythmia” or “2:1 phenotype.” The atrio-ventricular block can be recorded up to 7d post-fertilization, and the mutant phenotype is fully penetrant. Homozygous breakdance larvae appear wild-type with respect to all other morphological features and develop normally. Homozygous breakdance adults are viable and fertile.

The methods used to confirm that the breakdance mutant is defective in the zebrafish ortholog of HERG (ZERG) are further described in the Examples. Briefly, it was discovered that wild-type larvae treated with HERG-blocking (and QT-prolonging) drugs display the same 2:1 heartbeat ratio as seen in the breakdance larvae. Accordingly it was hypothesized that the breakdance mutant might correspond to a defect in a zebrafish HERG ortholog. Standard methods were used to map and clone the breakdance defect and the zebrafish HERG ortholog, and to produce the same phenotype using antisense oligonucleotides directed to ZERG, thus confirming that the breakdance phenotype is caused by a mutation in the ZERG gene. To our knowledge, this is the first demonstration of an “in organismo” knockdown of an ion channel, mimicking a channelopathy.

Nucleic Acids and Polypeptides of the Invention

As used herein, a teleost ERG gene refers to a teleost gene encoding an ERG family potassium channel (i.e., “a teleost ERG polypeptide”). A naturally occurring teleost ERG gene is endogenously expressed in the teleost larval heart; disruption of expression of the teleost ERG gene in a larva results in a cardiac phenotype selected from the group consisting of 2:1 arrhythmia, irrregular arrhythmia, bradycardia, aberrant heart morphology, blood accumulation in the yolk, and lack of circulation. Zebrafish ZERG is one example of a teleost ERG. ZERG nucleic acid (mRNA) and polypeptide sequences are provided, respectively, in SEQ ID NO:1 and in SEQ ID NO:2.

A teleost ERG is generally derived from a teleost organism or isolated cells or tissue thereof. However, it is understood that the same or similar sequences may be chemically synthesized and/or may be altered by human intervention (e.g., by introducing specific mutations that result in amino acid substitutions, additions or deletions, by introducing changes to codons that do not change the encoded amino acids, etc.). Such sequences that are produced or altered by human intervention are specifically included within the scope of teleost ERG genes. As used herein, the term “gene” refers to the segment of DNA involved in producing a polypeptide chain, which may or may not include regions preceding and following the coding region (e.g., 5′ UTR, 3′UTR, introns, promoter and enhancer sequences, etc.). The term “expression” refers to the process by which a polypeptide is produced based on the nucleic acid sequence of a gene; the process includes both transcription and translation.

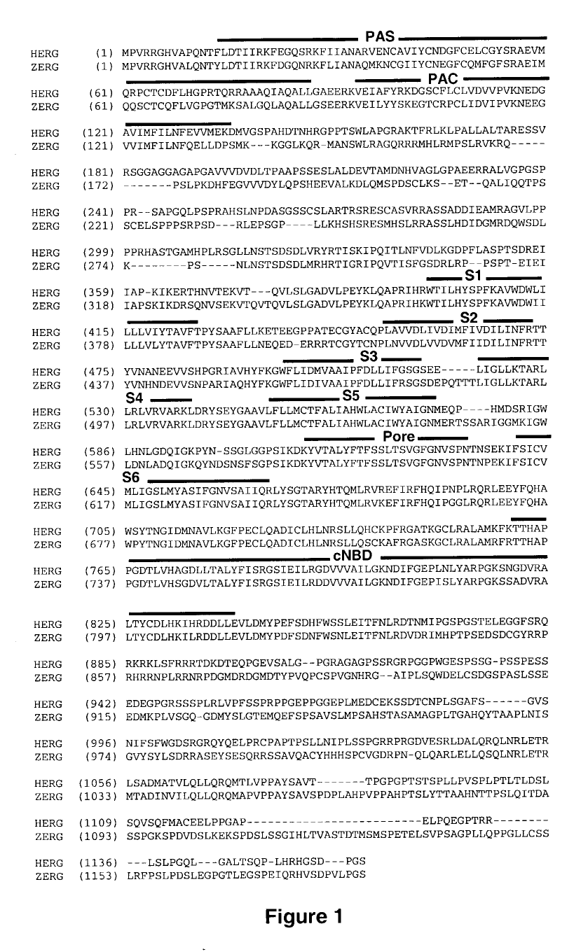

Methods of identifying the teleost orthologs of ZERG are known in the art. Normally, orthologs in different species retain the same function, due to presence of one or more protein motifs and/or 3-dimensional structures. In evolution, when a gene duplication event follows speciation, a single gene in one species, such as zebrafish, may correspond to multiple genes (paralogs) in another. As used herein, the term “orthologs” encompasses paralogs. When sequence data is available for a particular teleost species, orthologs are generally identified by sequence homology analysis, such as BLAST analysis, usually using protein bait sequences. Sequences are assigned as a potential ortholog if the best hit sequence from the forward BLAST result retrieves the original query sequence in the reverse BLAST (Huynen and Bork 1998; Huynen et al., 2000). A teleost gene is recognized as encoding an ERG-family potassium channel if, when the associated nucleic acid coding sequence (generally cDNA or niRNA but may include introns) or polypeptide sequence is subjected to BLAST analysis (preferably BLASTP, alternatively BLASTN, BLASTX, TBLASTN or TBLASTX), top hits are to other ERG family nucleic acids or polypeptides. Programs for multiple sequence alignment, such as CLUSTAL (Thompson et al, 1994) may be used to highlight conserved regions and/or residues of orthologous proteins and to generate phylogenetic trees. ERG nucleic acid and protein sequences from several vertebrate species, including human, mouse, dog, rabbit and chicken, are publicly available, and an alignment of HERG and ZERG proteins sequences, which was generated from a multisequence alignment using ERG sequences from rabbit, dog, and mouse as well, is provided in FIG. 1. In a phylogenetic tree representing multiple homologous sequences from diverse species (e.g., retrieved through BLAST analysis), orthologous sequences from two species generally appear closest on the tree with respect to all other sequences from these two species. Structural threading or other analysis of protein folding (e.g., using software by ProCeryon, Biosciences, Salzburg, Austria) may also identify potential orthologs. Nucleic acid hybridization methods may also be used to find orthologous genes and are preferred when sequence data are not available. Degenerate PCR and screening of cDNA or genomic DNA libraries are common methods for finding related gene sequences and are well known in the art (see, e.g., Sambrook, 1989; Dieffenbach and Dveksler, 1995). For instance, methods for generating a cDNA library from the teleost species of interest and probing the library with partially homologous gene probes are described in Sambrook et al. A highly conserved portion of the ZERG coding sequence (presented as nucleotides 99-3659 of SEQ ID NO:1) may be used as a probe. ZERG ortholog nucleic acids may hybridize to the nucleic acid of SEQ ID NO:1 under high, moderate, or low stringency conditions. After amplification or isolation of a segment of a putative ortholog, that segment may be cloned and sequenced by standard techniques and utilized as a probe to isolate a complete cDNA or genomic clone. Alternatively, it is possible to initiate an EST project to generate a database of sequence information for the teleost species of interest. In another approach, antibodies that specifically bind known ZERG polypeptides are used for ortholog isolation (see, e.g., Harlow and Lane, 1988, 1999). Western blot analysis can determine that a teleost ERG ortholog (i.e., an orthologous protein) is present in a crude extract of a particular teleost species. When reactivity is observed, the sequence encoding the candidate ortholog may be isolated by screening expression libraries representing the particular teleost species. Expression libraries can be constructed in a variety of commercially available vectors, including lambda gt11, as described in Sambrook, et al., 1989. Once the candidate ortholog(s) are identified by any of these means, candidate orthologous sequence are used as bait (the “query”) for the reverse BLAST against sequences from zebrafish or other species in which teleost ERG nucleic acid and/or polypeptide sequences have been identified.

As used herein, the term “teleost ERG polypeptide” refers to a full-length teleost ERG protein or a fragment, derivative (variant), or ortholog thereof that is “functionally active,” meaning that the protein fragment, derivative, or ortholog exhibits one or more or the functional activities associated with the polypeptide of SEQ ID NO:2. In one embodiment, a functionally active teleost ERG polypeptide is capable of rescuing defective (including deficient) endogenous teleost ERG activity when expressed in a teleost or in teleost cells; the rescuing polypeptide may be from the same or from a different species as that with defective activity. In another embodiment, a functionally active fragment of a full length teleost ERG polypeptide retains one of more of the biological properties associated with the full-length teleost ERG polypeptide, such as signaling activity, binding activity, catalytic activity, or cellular or extra-cellular localizing activity. Preferred teleost ERG polypeptides bind to co-factors. Other preferred teleost ERG polypeptides display ion channel activity. A teleost ERG fragment preferably comprises a teleost ERG domain, such as a C- or N-terminal or catalytic domain, among others, and preferably comprises at least 10, preferably at least 20, more preferably at least 25, and most preferably at least 50 contiguous amino acids of a teleost ERG protein.

Functional domains can be identified using the PFAM program (Bateman A et al., 1999; website at pfam.wustl.edu). A preferred teleost ERG fragment comprises a domain selected from the group consisting of a PAS (eag) domain, a PAC domain, a pore region, a cyclic nucleotide binding domain (cNBD), and a drug binding domain. Additional preferred fragments comprise the membrane-spanning domains. Descriptions of these various domains are publicly available (e.g., PFAM: Bateman et al., 1999; PSORT: Nakai and Horton, 1999, and Nakai, 2000; Mitcheson et al, 2000). For instance, descriptions of PAS, PAC, and cNBD domains are provided in, respectively PFAM accession numbers PF00989, PF00785, and PF00027. Preferred ZERG fragments comprise the following: the PAS (eag) domain, located at approximately amino acids 17-87 of SEQ ID NO:2, which are encoded by nucleotides 147-359 of SEQ ID NO:1; the PAC domain, located at approximately amino acids 93-135 of SEQ ID NO:2, which are encoded by nucleotides 375-503 of SEQ ID NO:1; the pore region, located at approximately amino acids 583-604 of SEQ ID NO:2, which are encoded by nucleotides 1845-1910 of SEQ ID NO:1; the cNBD, located at approximately amino acids 736-809 of SEQ ID NO:2, which are encoded by nucleotides 2304-2525 of SEQ ID NO:1; and the drug binding domain, located in the region of the “S6” domain, located at approximately amino acids 611-637 of SEQ ID NO:2, which are encoded by nucleotides 1929-2009 of SEQ ID NO:1. The ZERG putative membrane-spanning domains (S1-S6) are located at the following approximate positions: amino acids 361-387 of SEQ ID NO:2, which are encoded by nucleotides 1179-1259 of SEQ ID NO:1; amino acids 414-434 of SEQ ID NO:2, which are encoded by nucleotides 1338-1400 of SEQ ID NO:1; amino acids 459-476 of SEQ ID NO:2, which are encoded by nucleotides 1473-1526 of SEQ ID NO:1; amino acids 487-505 of SEQ ID NO:2, which are encoded by nucleotides 1557-1613 of SEQ ID NO:1; amino acids 516-539 of SEQ ID NO:2, which are encoded by nucleotides 1644-1715 of SEQ ID NO:1; and amino acids 611-637 of SEQ ID NO:2, which are encoded by nucleotides 1929-2009 of SEQ ID NO:1.

Functionally active variants of full-length teleost ERG polypeptides or fragments thereof include polypeptides with amino acid insertions, deletions, or substitutions that retain one of more of the biological properties associated with the full-length teleost ERG polypeptide. In some cases, variants are generated that change the post-translational processing of a teleost ERG polypeptide. For instance, variants may have altered protein transport or protein localization characteristics or altered protein half-life compared to the native polypeptide.

As used herein, the term “teleost ERG nucleic acid” encompasses nucleic acids (i.e., polynucleotides) with the sequence provided in or complementary to the sequence provided in SEQ ID NO:1 (ZERG), as well as functionally active fragments, derivatives, and orthologs thereof. A teleost ERG nucleic acid of this invention may be DNA, derived from genomic DNA or cDNA, or RNA.

In one embodiment, a functionally active teleost ERG nucleic acid encodes or is complementary to a nucleic acid that encodes a functionally active teleost ERG polypeptide. Included within this definition is genomic DNA that serves as a template for a primary RNA transcript (i.e., an mRNA precursor) that requires processing, such as splicing, before encoding the functionally active teleost ERG polypeptide. A teleost ERG nucleic acid can include other non-coding sequences, which may or may not be transcribed; such sequences include 5′ and 3′ UTRs, polyadenylation signals and regulatory sequences that control gene expression, among others, as are known in the art. Some polypeptides require processing events, such as proteolytic cleavage, covalent modification, etc., in order to become fully active. Accordingly, functionally active nucleic acids may encode the mature or the pre-processed teleost ERG polypeptide, or an intermediate form. A teleost ERG polynucleotide can also include heterologous coding sequences, for example, sequences that encode a marker included to facilitate the purification of the fused polypeptide, or a transformation marker.

A teleost ERG nucleic acid can also include non-coding sequences, such as 5′ and 3′ sequences transcribed, untranslated sequences, termination signals, ribosome binding sites, sequences that stabilize mRNA, introns, polyadenylation signals. A Teleost ERG nucleic acid can also include non-transcribed sequences that control gene expression, such as native promoters or enhancers.

When an isolated nucleic acid of the invention comprises a teleost ERG nucleic acid sequence flanked by non-ERG nucleic acid sequence, the total length of the combined nucleic acid is typically less than 25 kb, and usually less than 20 kb, or 15 kb, and in some cases less than 10 kb, or 5 kb.

In another embodiment, a functionally active teleost ERG nucleic acid is capable of being used in the generation of loss-of-function teleost ERG phenotypes, for instance, via antisense knock-down.

In one preferred embodiment, a teleost ERG nucleic acid of this invention is identified as a teleost nucleic acid sequence that encodes or is complementary to a sequence that encodes a teleost ERG polypeptide having at least 65%, 70%, 75%, 80%, 85%, 90%, 95% or more sequence identity to the ZERG polypeptide sequence presented in SEQ ID NO:2.

In another embodiment a teleost ERG polypeptide of the invention comprises a polypeptide sequence with at least 65% identity to the ZERG polypeptide sequence of SEQ ID NO:2, and may have at least 70%, 80%, 85%, 90% or 95% or more sequence identity to the ZERG polypeptide sequence of SEQ ID NO:2. In another embodiment, a teleost ERG polypeptide comprises a polypeptide sequence with at least 75%, 80%, 85%, 90% or 95% or more sequence identity to a functionally active fragment of the polypeptide presented in SEQ ID NO:2, and preferably comprises at least 75% sequence identity to the ZERG PAS domain or at least 90% identity to the ZERG cNBD domain. In yet another embodiment, a teleost ERG polypeptide comprises a polypeptide sequence with at least 65 %, 70%, 75%, 80%, 85% or 90% identity to the polypeptide sequence of SEQ ID NO:2 over its entire length and comprises a domain selected from the group consisting of a PAS domain, a PAC domain, a pore region, a cNBD, and a drug binding domain.

In another aspect, a teleost ERG polynucleotide sequence is at least 65% identical over its entire length to the ZERG coding sequence [cds] presented as nucleotides 99-3659 of SEQ ID NO:1, or nucleic acid sequences that are complementary to ZERG cds sequence, and may comprise at least 70%, 75%, 80%, 85%, 90% or 95% or more sequence identity to the ZERG cds.

As used herein, “percent (%) sequence identity” with respect to a specified subject sequence, or a specified portion thereof, is defined as the percentage of nucleotides or amino acids in the candidate derivative sequence identical with the nucleotides or amino acids in the subject sequence (or specified portion thereof), after aligning the sequences and introducing gaps, if necessary to achieve the maximum percent sequence identity, as generated by the program WU-BLAST-2.0a19 (Altschul et al. 1997; website at blast.wustl.edu/blast/README.html) with search parameters set to default values. The HSP S and HSP S2 parameters are dynamic values and are established by the program itself depending upon the composition of the particular sequence and composition of the particular database against which the sequence of interest is being searched. A “% identity value” is determined by the number of matching identical nucleotides or amino acids divided by the sequence length for which the percent identity is being reported. “Percent (%) amino acid sequence similarity” is determined by doing the same calculation as for determining % amino acid sequence identity, but including conservative amino acid substitutions in addition to identical amino acids in the computation. A conservative amino acid substitution is one in which an amino acid is substituted for another amino acid having similar properties such that the folding or activity of the protein is not significantly affected. Aromatic amino acids that can be substituted for each other are phenylalanine, tryptophan, and tyrosine; interchangeable hydrophobic amino acids are leucine, isoleucine, methionine, and valine; interchangeable polar amino acids are glutamine and asparagine; interchangeable basic amino acids are arginine, lysine and histidine; interchangeable acidic amino acids are aspartic acid and glutamic acid; and interchangeable small amino acids are alanine, serine, threonine, cysteine and glycine.

Teleost ERG nucleic acids may be identified as nucleic acids that selectively hybridize to the nucleic acid sequence of SEQ ID NO:1. The stringency of hybridization can be controlled by temperature, ionic strength, pH, and the presence of denaturing agents such as formamide during hybridization and washing. Conditions routinely used are well known (see, e.g., Ausubel et al., 1994 Current Protocol in Molecular Biology, Vol. 1, Chap. 2.10; Sambrook et al., 1989). In some embodiments, a nucleic acid molecule of the invention is capable of hybridizing to a nucleic acid molecule containing the nucleotide sequence of SEQ ID NO:1 under stringent hybridization conditions that comprise: prehybridization of filters containing nucleic acid for 8 hours to overnight at 65° C. in a solution comprising 6×single strength citrate (SSC) (1×SSC is 0.15 M NaCl, 0.015 M Na citrate; pH 7.0), 5×Denhardt's solution, 0.05% sodium pyrophosphate and 100 μg/ml herring sperm DNA; hybridization for 18-20 hours at 65° C. in a solution containing 6×SSC, 1×Denhardt's solution, 100 μg/ml yeast tRNA and 0.05% sodium pyrophosphate; and washing of filters at 65° C. for 1 h in a solution containing 0.2×SSC and 0.1% SDS (sodium dodecyl sulfate). In other embodiments, moderately stringent hybridization conditions are used that comprise: pretreatment of filters containing nucleic acid for 6 h at 40° C. in a solution containing 35% formamide, 5×SSC, 50 mM Tris-HCl (pH 7.5), 5 mM EDTA, 0.1% PVP, 0.1% Ficoll, 1% BSA, and 500 μg/ml denatured salmon sperm DNA; hybridization for 18-20 h at 40° C. in a solution containing 35% formamide, 5×SSC, 50 mM Tris-HCl (pH 7.5), 5 mM EDTA, 0.02% PVP, 0.02% Ficoll, 0.2% BSA, 100 μg/ml salmon sperm DNA, and 10% (wt/vol) dextran sulfate; followed by washing twice for 1 hour at 55° C. in a solution containing 2×SSC and 0.1% SDS. Alternatively, low stringency conditions can be used that comprise: incubation for 8 hours to overnight at 37° C. in a solution comprising 20% formamide, 5×SCC, 50 mM sodium phosphate (pH 7.6), 5×Denhardt's solution, 10% dextran sulfate, and 20 μg/ml denatured sheared salmon sperm DNA; hybridization in the same buffer for 18 to 20 hours; and washing of filters in 1×SSC at about 37° C. for 1 hour.

As a result of the degeneracy of the genetic code, a number of polynucleotide sequences encoding a ZERG polypeptide or another teleost ERG polypeptide can be produced. For example, codons may be selected to increase the rate at which expression of the polypeptide occurs in a particular host species, in accordance with the optimum codon usage dictated by the particular host organism (see, e.g., Nakamura et al, 1999). Such sequence variants may be used in the methods of this invention.

In a preferred embodiment, ZERG or another teleost ERG is engineered to incorporate mutations corresponding to mutations in BERG gene that cause prolonged QT (Itoh et al. 1998; Splawski et al., 2000; January et al., 2000; Vatta et al., 2000).

In another preferred embodiment, a chimeric or hybrid teleost ERG gene can be constructed. An exemplary hybrid gene comprises the human HERG gene under control of the ZERG regulatory sequences or under control of a cardiac promoter (see, e.g., Rothman et al., 1996; Prentice et al. 1997; Franz et al. 1994). The HERG amino acid sequence is provided in SEQ ID NO:5. An exemplary chimeric gene comprises coding sequences (i.e., encoding particular amino acid residues) derived from both ZERG and HERG genes, which are typically under control of a cardiac associated promoter. For instance, a chimeric gene may comprise primarily HERG sequences, but may have particular ZERG residues substituted to increase the stability or function of the protein. Alternatively, a chimeric gene comprises primarily ZERG sequences, but has particular HERG residues substituted to more closely mimic HERG interaction with drugs. As one skilled in the art will appreciate, a sequence alignment of multiple ERG sequences, such as that provided in FIG. 1, will indicate corresponding residues and regions and thus provide guidance in making specific sequence replacements. In specific embodiments, a chimeric gene encodes a ZERG derivative polypeptide wherein one or more residues or fragments presented in Table 1, first column, have been replaced with the corresponding residues or fragments from HERG, shown in the second column of Table 1.

TABLE 1

| Amino acid | Amino acid | |
|---|---|---|
| residue(s) from ZERG | residue(s) from HERG | |
| (SEQ ID NO:2) | (SEQ ID NO:5) | Region |
| 376 (Ile) | 413 (Leu) | S1 domain |
| 388-413 | 425-451 | Intervening S1 and S2 |
| | | domains |
| 414-434 | 452-472 | S2 domain |
| 435-458 | 437-496 | Intervening S2 and S3 |
| | | domains |
| 476 (Arg) | 514 (Gly) | S3 domain |
| 540-575 | 573-603 | Intervening S5 and pore |
| | | domains |
| 608 (Pro) | 636 (Ser) | Intervening pore and S6 |
| | | domains |

As used herein, when it is said that a chimeric gene encodes a chimeric polypeptide comprising an amino acid sequence “derived from” a particular sequence (e.g., the ZERG sequence of SEQ ID NO:2), it is meant that the chimeric polypeptide is identical to that particular sequence in all residues except those residues that were specifically replaced. In one embodiment, the chimeric ZERG gene encodes the chimeric polypeptide whose sequence is presented in SEQ ID NO:4, where the entire membrane-associated region from HERG has replaced the corresponding ZERG region. A transgenic teleost comprising such hybrid or chimeric genes is termed a “humanized teleost.” This animal may be one in which the chimeric gene was directly introduced, or may be the direct or indirect progeny of such a transformed animal. Furthermore, the humanized teleost may have wild-type teleost ERG alleles, or may contain a mutant ERG gene. The portions of a chimeric or hybrid gene that encode non-teleost residues may comprise codon sequences native to the non-teleost gene (e.g., HERG) or may comprise codon sequences optimized for expression in the teleost host. Various methods for humanizing non-human genes for introduction into non-human species are known in the art (see, e.g., Reaume et al., 1996; Muldoon et al., 1997).

An isolated teleost ERG nucleic acid molecule is other than in the form or setting in which it is found in nature and is identified and separated from least one contaminant nucleic acid molecule with which it is ordinarily associated in the natural source of the teleost ERG nucleic acid. However, an isolated teleost ERG nucleic acid molecule includes teleost ERG nucleic acid molecules contained in cells that ordinarily express teleost ERG where, for example, the nucleic acid molecule is in a chromosomal location different from that of natural cells.

Isolation, Production, Expression and Mis-expression of Teleost ERG Nucleic Acids and Polypeptides

Teleost ERG nucleic acids and polypeptides may be obtained using methods that are well known to those of skill in the art. For instance, techniques for isolating cDNA or genomic DNA sequences of interest by screening DNA libraries or by using polymerase chain reaction (PCR) are well known in the art.

A wide variety of methods are available for obtaining ZERG polypeptides. In general, the intended use for the polypeptide will dictate the particulars of expression, production, and purification methods. For instance, overexpression of a ZERG polypeptide for cell-based electrophysiology assays may require expression in eukaryotic cell lines amenable to electrophysiology. Techniques for the expression, production, and purification of proteins are well known in the art; any suitable means therefor may be used (e.g., Higgins and Hames, 1999; Coligan et al, 1999; U.S. Pat. No. 6,165,992).

The nucleotide sequence encoding a teleost ERG polypeptide can be inserted into any appropriate vector for expression of the inserted protein-coding sequence. The necessary transcriptional and translational signals, including promoter/enhancer element, can derive from a native teleost ERG gene and/or its flanking regions or can be heterologous. A variety of host-vector expression systems may be utilized, such as mammalian cell systems infected with virus (e.g. vaccinia virus, adenovirus, etc.); insect cell systems infected with virus (e.g. baculovirus); microorganisms such as yeast containing yeast vectors, or bacteria transformed with bacteriophage, plasmid, or cosmid DNA. A host cell strain that modulates the expression of, modifies, and/or specifically processes the gene product may be used.

The teleost ERG polypeptide may be optionally expressed as a fusion or chimeric product, joined via a peptide bond to a heterologous protein sequence. A chimeric product can be made by ligating the appropriate nucleic acid sequences encoding the desired amino acid sequences to each other in the proper coding frame using standard methods and expressing the chimeric product. A chimeric product may also be made by protein synthetic techniques, e.g. by use of a peptide synthesizer (Hunkapiller et al., 1984).

A teleost ERG polypeptide can be isolated and purified using standard methods (e.g. ion exchange, affinity, and gel exclusion chromatography; centrifugation; differential solubility; electrophoresis). Alternatively, native teleost ERG proteins can be purified from natural sources, by standard methods (e.g. immunoaffinity purification). Once a protein is obtained, it may be quantified and its activity measured by appropriate methods, such as immunoassay, bioassay, or other measurements of physical properties, such as crystallography.

The methods of this invention may use cells that have been engineered for altered expression (mis-expression) of teleost ERG. As used herein, mis-expression encompasses ectopic expression, over-expression, under-expression, and non-expression (e.g. by gene knock-out or blocking expression that would otherwise normally occur).

Nucleic Acid Inhibitors

The present invention provides methods for inhibiting the function of an endogenous teleost ERG gene using specific nucleic acid inhibitors. The nucleic acid inhibitor can be DNA, RNA, a chimeric mixture of DNA and RNA, derivatives or modified versions thereof, single-stranded or double-stranded. In one embodiment, the inhibitor is a ZERG-specific antisense oligomer, preferably of length ranging from at least 6 to about 200 nucleotides. The oligomer can be modified at the base moiety, sugar moiety, or phosphate backbone. In a preferred embodiment, the antisense oligomer is sufficiently complementary to a teleost ERG to bind to the teleost ERG mRNA and prevent translation. As used herein, an antisense oligomer is said to be “capable of inactivating” a specific gene if administration of the oligomer under suitable conditions disrupts the normal expression of the gene and causes a loss-of-function phenotype; the antisense oligomer generally inhibits translation of the transcript. Oligomers that partially disrupt gene expression and/or cause partial loss-of-function phenotypes are included in this definition.

In a preferred embodiment, the antisense oligomer is a phosphorothioate morpholino oligonucleotide (PMO). PMOs are assembled from four different morpholino subunits, each of which contains one of four genetic bases (A, C, G, or T) linked to a six-membered morpholine ring. Non-ionic phosphodiamidate intersubunit linkages join polymers of these subunits. Methods of producing and using PMOs and other antisense oligomers are well known in the art (e.g., Probst, 2000; Summerton and Weller 1997; U.S. Pat. Nos: 5,235,033 and 5,378,841).

Methods for gene inactivation in zebrafish using PMOs are well known in the art (Nasevicius and Ekker, 2000). PMOs of this invention are approximately 10-50 nucleotides, preferably approximately 15-40 nucleotides, preferably 20-30 nucleotides, and most preferably 21-25 nucleotides. Preferred PMOs may be directed to the 5′ end of a teleost ERG gene such that they cover or lie upstream of the start codon. Alternative preferred PMOs may be directed to splice junctions, preferably to exon-intron boundaries (Draper et al., 2001; Schmajuk et al., 1999). Methods for obtaining the genomic DNA sequence corresponding to specific mRNA sequences and for determining the intron-exon boundaries by comparing genomic DNA and mRNA sequences are well known in the art.

In one embodiment, preferred PMOs comprise a sequence complementary to contiguous nucleotides within nucleotides 1-150 of SEQ ID NO:1; an exemplary PMO sequence is presented in SEQ ID NO:3. As further detailed in the Examples, we generated an antisense PMO corresponding to the first 24 nucleotides of the ZERG coding sequence, as presented in SEQ ID NO:3, and injected these into the yolk of zebrafish embryos at the 1-4 cell stage, according to standard protocols. About 90% of the PMO-injected larvae displayed the 2:1 phenotype, larvae otherwise not distinguishable from wild type larvae. Thus, antisense PMOs may be used to knockdown the ZERG protein, and to phenocopy the breakdance mutant, as well as QT-prolonging drug-treated larvae.

A variety of other antisense reagents may be used to inactivate teleost ERG genes. For instance, a preferred antisense oligomer is peptide nucleic acid, (PNA) a nucleic acid analog with an achiral polyamide backbone (Soomets et al., 1999). They concluded that M (Modified PNAs have been used for gene inactivation in zebrafish and have been shown to have comparable potency and higher specificity than PMOs (Urtishak et al., 2002). Other preferred antisense oligomers have been modified for delivery, for instance by annealing to blocking nucleic acid molecules (e.g., PCT application WO0234908).

Alternative nucleic acid inhibitors are double stranded RNA duplexes, or “small interfering RNAs” (Elbashir et al., 2000).

Genetically Modified Animals

The methods of this invention may use non-human animals, preferably teleosts, which have been genetically modified (i.e., genetically engineered) to alter expression of ZERG or another teleost ERG, or chimeric, hybrid, or humanized teleost ERG genes. In a preferred embodiment, such genetic modification results in a cardiac phenotype; exemplary cardiac phenotypes are further described below.

Preferred genetically modified animals are transgenic, at least a portion of their cells harboring non-native nucleic acid that is present either as a stable genomic insertion or as an extra-chromosomal element, which is typically mosaic. Preferred transgenic animals have germ-line insertions that are stably transmitted to all cells of progeny animals.

For production of transgenic teleosts, non-native nucleic acid is introduced into host animals by any expedient method. Methods for producing transgenic zebrafish are well known in the art (see, e.g., Culp et al., 1991; Lin, 2000; Koster R W, Fraser S E, 2001; Hsiao et al, 1999; Linney E, 2001; Ju et al., 1999). Methods for producing transgenic medaka are also well known (see, e.g., Tanaka et al, 2001; Takagi et al., 1994; Ozato et al., 1986). Methods for producing germ-line chimeras from embryo cell cultures have been developed for both zebrafish (Ma et al., 2001) and medaka (Hong et al., 1998), and the generation of fertile, diploid adults from nuclear transplantation has been accomplished in medaka (Wakamatsu et al., 2001). Methods for homologous recombination are available in various non-human organisms and cells (e.g., Capecchi, 1989; Joyner et al., 1989; Rong and Golic, 2000; Mateyak et al., 1997; Francès and Bastin, 1996).

Homozygous or heterozygous alterations in the genomes of transgenic animals may result in mis-expression of native genes, including ectopic expression, over-expression (e.g. by multiple gene copies), under-expression, and non-expression (e.g. by gene knock-out or blocking expression that would otherwise normally occur). In one application, a loss-of-function animal is generated, typically using homologous recombination, in which an alteration in an endogenous gene causes a decrease in that gene's function. A “knock-out” animal may be generated such that gene expression is undetectable or insignificant. In another application, ectopic expression is produced by operatively inserting regulatory sequences, including inducible, tissue-specific, and constitutive promoters and enhancer elements, to direct altered spatial and/or temporal expression of an endogenous gene. Transgenic, nonhuman animals can also be produced using systems that provide regulated expression of the transgene, such as the cre/loxP (Lakso et al., PNAS (1992) 89:6232-6236; U.S. Pat. No. 4,959,317) and FLP/FRT (O'Gorman et al. (1991) Science 251:1351-1355; U.S. Pat. No. 5,654,182) recombinase systems.

Alternatively, additional teleost ERG mutations can be isolated using non-targeted (random) mutagenesis techniques, for instance, chemical-, X-ray, or transposon mutagenesis (e.g., Chen et al., 1996; Kawakami et al., 2000).

Also included with in the scope of genetically modified animals are teleosts, including teleost larvae, in which expression of the endogenous teleost ERG gene has been specifically disrupted by administration of an antisense oligomer comprising sequences complementary to the endogenous ERG gene.

Teleost Models of Cardiac Function

The breakdance larvae and the methods of this invention provide a teleost model for inherited HERG-blockade (Long-QT2-disease), which may be used for the study of HERG blockade, atrioventricular block, arrhythmia and the Long-QT-syndrome. The invention provides methods for testing for the cardiac activity of pharmaceutically active agents using a teleost ERG gene, teleosts containing mutations in teleost ERG genes, and nucleic acid inhibitors that target teleost ERG genes. Such methods generally comprise 1) providing teleost larvae (wild type or mutant) in a suitable medium and in an appropriate screening format, 2) contacting the teleost larvae with a candidate agent, and 3) detecting phenotypic changes produced by the candidate agent. As further described below, exemplary applications of these methods include screens for anti-arrhythmic agents that revert the 2:1 phenotype of mutant teleost having reduced teleost ERG activity and screening for candidate drugs that produce unwanted arrhythmias. As used herein, the term “mutant teleost larvae having reduced teleost ERG activity” is used to encompass genetically wild-type larvae treated with specific nucleic acid inhibitors such as PMOs or other teleost ERG inhibitors, as well as teleosts carrying mutations in an endogenous teleost ERG gene. Such larvae will generally display a visually detectable cardiac phenotype. An agent capable of producing a cardiac phenotype in teleost larvae is referred to as a cardio-active agent; exemplary cardiac phenotypes are further described below. Screening methods of this invention involve comparing the cardiac phenotype of teleosts (either wild-type or mutant) in the presence and absence of treatment with candidate agents. If an agent changes the cardiac phenotype of the subject teleost larvae, it is said to produce an “agent-biased phenotype.”

The methods of this invention may be used to test the effect of many different kinds of pharmaceutically active agents (see, e.g., WO9942606). Preferred agents are small molecule compounds, which are typically organic, non-peptide molecules, having a molecular weight less than 10,000, preferably less than 5,000, more preferably less than 1,000, more preferably less than 750, and most preferably less than 500. This class of agents includes chemically synthesized molecules, for instance, compounds from combinatorial chemical libraries. Small molecule compounds also include natural products, particularly secondary metabolites from organisms such as plants or fungi.

Agents may be applied using any expedient method, such as bathing, injection, feeding, etc. In a preferred embodiment, the teleost larvae are incubated in a suitable medium, preferably for about 2-7days at about 22-28° C. Suitable media for raising teleost alevin are known in the art and include low salt, buffer solutions (e.g., solutions containing less than 10 mM salts [alkaline and earth alkaline salts] and less than 20 mM buffer substance). A preferred medium for zebrafish larvae is “embryo medium,” comprising:4.9 mM NaCl, 170 μM KCl, 329 μM CaCl₂, 331 μM MgSO₄, pH 7.2 (Westerfield, 1993), which is supplemented with 10 mM HEPES.

In one application, pharmaceutically active agents are added to the medium preferably 2-5 days after fertilization of the teleost alevin. If the agent is a small molecule compound, it is preferably added at concentration of 100 nM to 100 μM, most preferably at 1-100 μM. The media may include up to 0.5% dimethyl sulfoxide (DMSO), which is tolerated by teleost larvae, in order to enhance compound penetrance. Other agents, such as transfection reagents, may further stimulate uptake. Agents may alternatively be injected, for instance, near the sinus venosus, into the artery, or into the yolk sac of 1-4 cell stage larvae. Injection may be preferred if the agents do not diffuse into the larvae, for instance, due to low lipophilicity (since compounds are thought to enter the larvae via the skin, crossing lipid-rich membranes, lipophobic compounds may not easily enter the larvae). When screening methods use PMO-treated larvae, agents may also be co-injected with the PMOs. This method may render the larvae more sensitive to cardio-active agents drugs and obviates the requirement that the agent penetrate the skin. Injection of compounds may also be preferred when later-stage larvae, whose skin is less permeable, are used.

Cardiac function may be assessed using any expedient detection methods. In a preferred embodiment, aberrant cardiac function is detected via visual inspection. In an alternative method, which may be preferred when large numbers of agents are tested, a video capture system records heart appearance and function (e.g., Schwerte and Pelster, 2000). Alternatively, fluorescent dyes, such as ANEPPS or Fura, may be used to detect membrane potential and cytosolic calcium changes in zebrafish heart (e.g., as demonstrated using intact guinea pig heart, Laurita and Singal, 2001). Electrophysiological methods, such as electrocardiogram (ECG) readings, may also be used to monitor heartbeat.

In a preferred embodiment, heart beat (rate, rhythm and contractility) and blood flow are visually monitored, for instance, using a dissecting or other microscope, within two hours after addition of the agent. The 2:1 phenotype is easily recognized by visual inspection. In order to quantify the response, for each teleost analyzed, heart beat rate of each chamber is counted with the help of a timer. When screening for agents that ameliorate or exacerbate the 2:1 arrhythmia, percentage of larvae displaying the 2:1 heat beat, or the percentage of rescued larvae (rescue from 2:1 arrhythmia to a 1:1 heart beat) is also recorded. The readout, the 2:1 arrhythmia, is easily detected (for comparison, in vivo ECG measurements must detect increases in the QT-interval of a few milliseconds following application of QT-prolonging drugs). Other abnormal cardiac phenotypes that can be easily detected via visual inspection include heart morphology, such as cardiomyopathy, lack of circulation, and blood accumulation in the yolk.

In some applications, the methods of this invention will use wild-type larvae treated with QT-prolonging drugs (e.g., as a control or reference). In this case, wild-type teleost larvae are incubated with QT-prolonging drugs and the pharmaceutically active agents to be tested are added either subsequently or simultaneously.

With methods of the present invention, teleosts can be used to screen a large number of compounds for their effects on heartbeat. For example, using 24 well format and manual techniques for addition of the drug, pipetting of the larvae, microscopy, etc., about 300 substances per day and person can be tested for their effects on the heart (assuming two concentrations per compound tested, and approximately ten teleost larvae per well). Methods for increased throughput that rely on, for instance, automated fluid and micro-plate manipulations, have been developed (see, e.g., PCT publication WO9942606). It has been shown that zebrafish larvae can be maintained in a standard 96-well plate format, in as little as 100 μl fluid through the first six days of development.

A particular advantage of the disclosed methods for detecting cardio-active agents, in comparison to cell-free or cell-based conventional in vitro HTS assays, is that the agents tested act on an intact heart integrated in the whole-body physiology. Compared to studies in rodents or other mammals, studies with teleost larvae are significantly simpler, faster, and less costly. In one application, compounds identified through high throughput screening assays may be prioritized using the teleost assays, before more complicated validation experiments involving, for example, mammals such as mice or rats.

In one application of the methods disclosed herein, wild type teleost larvae are used to screen for candidate drugs that may produce unwanted arrhythmias. As further described in the Examples, zebrafish larvae are quite sensitive towards QT-prolonging drugs. We have found zebrafish larvae bathed in media containing various QT-prolonging drugs showed the 2:1 arrhythmia previously described for the breakdance larvae. The cloning of ZERG and the discovery that the same phenotype produced by HERG-blocking drugs is caused by a mutant ZERG gene provides a rational basis for performing drug testing in teleosts. Given that a wide variety of compounds act as HERG-blocking agents, this discovery provides for the development of a valuable assay for the early detection of drugs that might promote acquired QT disease. In a similar application, wild type larvae can be used to screen for specific inhibitors of HERG channels; such inhibitors have become an important tool for the maintenance of sinus rhythm in patients with arterial flutter and atrial fibrillation (Mounsey and DiMarco, 2000).

In another application, “humanized” teleosts, which express the human HERG gene or a “HERG/teleost ERG ” hybrid gene, comprising one or more HERG amino acid residues and one or more amino acid residues from a teleost ERG, are used for screening for HERG-blocking agents that induce acquired long-QT syndrome. Such teleosts preferably have reduced or absent native teleost ERG activity. Exemplary humanized teleosts are breakdance zebrafish that are genetically modified to express the HERG gene or a HERG/ZERG hybrid under control of ZERG regulatory sequences or another cardiac promoter. Preferably, the introduction of functional HERG or the HERG/ZERG hybrid rescues the 2:1 phenotype or other aberrant cardiac phenotypes associated with ZERG loss-of-function. In this application, screening identifies agents that cause the 2:1 phenotype or other phenotypes associated with ZERG loss-of-function. Such methods advantageously combine directly testing human BERG sensitivity to putative QT-prolonging agents with the simple readout afforded by the teleost assay.

In a preferred embodiment, the methods of this invention are used to screen for agents that can rescue the ZERG-induced arrhythmia by reverting the 2:1 arrhythmia to a normal heartbeat. Mutant zebrafish larvae that have reduced ZERG activity and that display the 2:1 phenotype are tested with pharmaceutically active agents in order to identify those that revert the 2:1 phenotype; similar experiments may be performed with other teleost larvae having reduced teleost ERG activity. Such agents may be useful in the development of specific anti-arrhythmic drugs for the treatment of QT syndrome (Tseng GN, 2001). Currently, common treatment of QT syndrome uses relatively unspecific cardiovascular drugs. ZERG also appears to regulate pacemaker activity and contractility, as further described in the Examples. Additionally, there have been observations that long QT syndrome can be associated with bradycardia (low heart beat rate) (e.g., Yoshida H et al., 2001). Accordingly, drugs that revert the 2:1 phenotype may also be useful for the treatment of bradycardia, congestive heart failure, and cardiomyopathies.

In another embodiment, the methods of the invention are used to screen for QT-prolonging drugs, and for drugs whose pro-arrhythmic activity is enhanced in animals containing mutations in teleost ERG genes. In this application, teleosts with reduced teleost ERG function provide a sensitized background. In one application, mutant animals with ZERG loss-of-function, particularly partial loss-of-function, provide the sensitized background. If these animals, such as breakdance larvae, already display a fully penetrant 2:1 phenotype, screening identifies other phenotypes, including irregular arrhythmia and heart beat rate and bradycardia. It is likely that the breakdance mutation is a partial loss-of-function mutation, since treating wild-type larvae with high concentrations of ZERG-specific PMOs or HERG-blocking drugs produce stronger cardiac phenotypes than observed in breakdance larvae. It also might be possible that the breakdance mutant or other partial loss-of-function larvae have increased sensitivity to QT-prolonging drugs, such that lower concentrations of QT-prolonging drugs may cause “real arrhythmia” (i.e., both chambers beating irregularly, also referred to as “irregular arrhythmia”) or bradycardia. Other possible phenotypes include aberrant heart morphology, such as cardiomyopathy, lack of circulation, and blood accumulation in the yolk. Alternatively, animals with partial loss-of-function of teleost ERG, which do not display the fully penetrant 2:1 phenotype, provide the sensitized background, and the screening identifies agents that produce the 2:1 phenotype. Such animals may show, for example, slight bradycardia or even no phenotype. Exemplary animals for use in such experiments include teleost larvae with engineered mutations in a teleost ERG or animals treated with a low dose of specific antisense oligomers. For instance, as described in the Examples, a lower dose of the ZERG PMO produced more mild cardiac phenotypes. In one preferred application, these methods are used as a secondary screen to test the cardio-active properties of agents that have been previously identified in another context (e.g., using high throughput screening). The advantage is that this method enables the early identification of pro-arrhythmic drugs and thus minimizes the risk that at a late stage drug development has to be stopped.

Teleost ERG genes may also be used for cell-based studies using Xenopus oocytes or cultured mammalian cells. Currently, some companies test for QT-prolonging effects of candidate drugs by measuring their effects on HERG channel currents, using electrophysiological and/or dye methods and HERG expressed in oocytes or mammalian cells. We observed that ZERG may be more sensitive towards some blockers than mammalian ERG genes. For instance, in the case of the drugs astemizole and terfenadine, the concentrations required to produce the 2:1 phenotype in zebrafish larvae were lower than were required to produce comparable effects in dogs (see Example 1; Gintant et al., 2001; Yamamoto et al., 2001).

Utility of Teleost ERG in Disease Research

The teleost ERG genes and teleosts comprising mutations therein may have further utility in non-cardiac research and pharmaceutical development.

Teleost ERG genes may be useful in the development of treatment for tumors. The HERG gene is preferentially expressed in tumor cells (Bianchi et al., 1998; Cherubini et al., 2000). Accordingly, cardio-active agents (e.g., HERG blockers) discovered by the methods of this invention may be useful for the treatment of tumors. The methods of this invention that use teleost ERG in screening for QT-prolonging drugs may be further useful for developing agents for cancer therapies. HERG blockers designed as therapeutic agents for the treatment of tumors would need to be designed to act specifically on the tumor, to avoid provoking lethal arrhythmias in treated patients.

Teleost ERG genes and teleosts comprising mutations therein may also be useful in brain and nervous system research. BERG and HERG-like genes are expressed in the mammalian nervous system cells, including astrocytes, microglia, and neurons (e.g., Emmi A et al., 2000; Eder C, 1998; Saganich M J et al., 2001; Tinel N et al., 2000; Bauer C K et al, 1998). As further described in the examples, we have shown that ZERG is expressed in zebrafish brain. Loss of function ZERG fish may be useful in elucidating the function of HERG in the brain and for the treatment of neurological diseases resulting from impaired HERG function.

Teleost ERGs gene and teleosts comprising mutations therein may further be useful in the study of insulin-related diseases. HERG is expressed in the pancreatic islet cells and may have a crucial role in regulating insulin secretion and firing of human beta-cells (Rosati et al., 2000).

All references cited herein, including publications, patents, patent applications, and gene and sequence data accessible through the Genbank identifier numbers and websites provided, are expressly incorporated by reference in their entireties.

The invention is further explained by the following non-limiting examples.

### EXAMPLES

### Example 1

### Pharmacological Studies Using Zebrafish Larvae

Zebrafish larvae were raised according to established protocols (M. Westerfield, The zebrafish book, University of Oregon Press, Eugene, Oreg., USA (1993).

About 50 zebrafish larvae were incubated for 2-7 d at 22-28° C. in petri dishes filled with 30 ml embryo medium.

At the day of the experiment 10 larvae were transferred to small petri dishes filled with 10 ml embryo medium, 0.5 ml MESAB solution (0.2% ethyl-m-aminobenzoate methane-sulfonate+1% Na₂HPO₄×2H₂O, pH 7.2) and 10 μl of the respective drug (or +10 μl of a second drug) added from a 1000-fold stock solution prepared with DMSO, these three components were previously mixed in 50 ml plastic tubes (controls only receive 10 or 20 μl DMSO). Drugs tested included terfenadine (10 μM), astemizol (1 μM), diphenhydramine (100 μM), haloperidol (2 μM), droperidol (50 μM), thioridazin (10 μM), pimozide (0.5 μM), propafenone (100 μM), tetracain (20 μM), lidocain (100 μM), orphenadrine (100 μM; Sigma); cisapride (5 μM; Research Diagnostics, Flanders N.J.), E-4031 (40 μM; Alomone Labs, Jerusalem) and epinastine (100 μM; Boehringer Ingelheim, Biberach).

At certain time points the contractility of the heart and blood flow was monitored, and the heart rate was determined by counting the heart beat with the aid of a stereo-microscope.

In order to evaluate if zebrafish is a suitable model organism to study drug-induced QT-prolongation we investigated the effect of known QT-prolonging medicaments on the heart beat rate of zebrafish larvae. Compounds were added to the media in which the larvae were bathed and the heart beat rate was recorded by microscopy. We found that several structurally unrelated compounds (the antihistamines astemizole, terfenadine and diphenhydramine, the prokinetic gastorintestinal drug cisapride, the antipsychotics pimozide, thioridazine, droperidol and haloperidol, respectively) induced a 2:1 atrioventricular block in zebrafish larvae. This means the atrium beats twice while the ventricle beats once, both chambers beating regularly. In the control fish the heart beats regularly and coordinately with a 1:1 ratio of atrial and ventricular beat.

The mean heart beat rate of control larvae was 148+/−10 beats/min. With the exception of astemizole and cisapride (at the respective concentrations), all other drugs induced a slight bradycardia after 1 h of incubation and the respective concentration, the beat rate of the atrium being reduced by about 20-30% of control. This indicates that the QT-prolonging drugs affect sinus as well as atrio-ventricular node activities. The safe antihistamine epinastine was used as a control and did not affect heart beat rate up to 250 μM. E-4031, a widely used HERG blocker in electrophysiological studies induced a similar response in the zebrafish larvae as the QT-prolonging drug. For tetracaine and orphenadrine, HERG blockade has not been published.

The lower the concentrations of the drugs were, the later the 2:1 heart beat occured (100 nM astemizole or 200 nM pimozide, 6 and 3 h incubation, respectively). In contrast, relatively high concentrations (e.g. 10 μM haloperidol or 1 μM pimozide) caused an atrioventricular block in less than 5 min of incuabation with the drug.

Increasing the concentrations of arrhythmia-inducing drugs or prolonging the incubation time often induced a stronger bradycardia of both chambers, the ventricle being more affected than the atrium. Simultaneously, the contractility of both chambers decreased markedly; often the block disappeared during this phase, both chambers then again beating coordinately 1:1. In the extreme case QT-prolonging drugs could also induce the arrest of the atrial and ventricular beat (25 μM cisapride or 10 μM pimozide, 1 hour incubation). No other effects of the drugs on the zebrafish larvae were observed.

With some treatments “real” arrhythmia occurred after bathing the larvae for a relatively long time (6 hours) with QT-prolonging drugs (e.g. 200 nM astemizol, 10 μM thioridazine, haloperidol or cisapride) the atrium and ventricle beating very irregularly, however, the atrium still beating more often than the ventricle.

In most cases the larvae could be rescued from the 2:1 block and the bradycardia to the control heart beat rate just by bathing the fish in drug-free solutions (for 30 min to several hours).

### Example 2

### Isolation of the ZERG Gene

In the large genetic zebrafish ENUJ screen performed in Tübingen 1996, (Chen et al., 1996) a mutant (breakdance, bre, tb218) characterized by an abnormal beating pattern of the heart was identified. Breakdance showed a 2:1 beat ratio, such that the ventricle contracts once while the atrium contracts twice.

Wild-type larvae, treated with QT-prolonging drugs are the exact phenocopy of the breakdance mutant. The pharmacological data support the suggestion that the breakdance mutant might be defective in a zebrafish ortholog of HERG. To prove this, we cloned the breakdance mutant.

In a genome wide bulk segregant analysis using simple sequence length polymorphism (SSLP) the breakdance locus was assigned to linkage group 3. Fine mapping using additional SSLP marker from this region mapped the locus to an interval of 1.88 cM between SSLP marker Z10934 and Z59122. Four recombinations in 584 meioses were found for Z10934, and 7 recombinations in 584 meioses for Z59122. In order to test the hypothesis of breakdance being caused by a mutation in the ZERG gene, a 384 bp fragment of the zebrafish ZERG gene was isolated by a reverse transcription-polymerase chain reaction (RT-PCR) approach using degenerate primers derived from Tetraodon viridis ESTs This fragment was subsequently used to map the ZERG locus on a radiation hybrid panel anchored to the genetic map. The ZERG gene locus maps to the same interval as the breakdance mutation.

The full length ZERG cDNA was isolated by 5′ and 3′ rapid amplification of cDNA ends (RACE) and is provided in SEQ ID NO:1. The cDNA sequence contains a single open reading frame encoding a predicted protein of 1186 amino acids with a predicted molecular mass of 132.3 kDa. The ATG at position 99 was used as the putative start codon, because it is the first ATG following an in frame stop codon at position 93. A potential polyadenylation signal was found at position 4571-4576. The predicted ZERG protein showed 59% identity and 69% similarity to HERG. ZERG also shows a high similarity to other mammalian and also chicken ERG channels (Mus musculus GI 2645991, Rattus norvegicus GI 2745729, Oryctolagus cuniculus GI 2351698, Canis familiaris GI 2407213, Gallus gallus GI 6706732). Like other ERGs, ZERG also contained 6 putative transmembrane domains and one pore region. Furthermore, a PAC domain and a cyclic-nucleotide-binding region, was identified, both shown to regulate activity of mammalian ERG channels. Several putative phosphorylation, myristylation and glycosylation sites can be found in ZERG, similar to HERG. In the pore region and drug-binding domains similarity to HERG is nearly 100% on an amino acid level. Amino acids shown to be involved in the binding of QT-prolonging drugs are all identical in zebrafish and human ERG.

To investigate whether or not in ZERG is responsible for the breakdance phenotype, we searched for mutations in the ZERG gene of homozygous breakdance fish. The strategy chosen consisted of direct sequencing of uncloned RT PCR fragments covering the whole open reading frame. An ATC to AGC exchange at position 176 of the open reading frame leading to an amino acid exchange from Isoleucine to Serine (codon 59), specific for the mutant fish, was identified. With the exception of Tetraodon viridis, which also has an isoleucine at this position, a valine can be found in all other species at this position, indicating a high conservation of a hydrophobic amino acid. The amino acid exchange lies within the PAS domain, shown to be important for controlling the rate of HERG deactivation (Sansom Miss., 1999).

The mutation identified was used to assess the segregation of the ZERG locus relative to the breakdance mutation. No recombination could be detected in the analyzed 142 meioses.

ZERG is not published in the database. One zebrafish EST with homology to ZERG, AW454917, is available not but not annotated.

### Example 3

### Generation of an Antisense ZERG-knockdown Model

24-mer antisense PMOs targeting the 5′ prime region of ZERG (SEQ ID NO:3; start 0-24: GAC ATG TCC GCG GCG CAC GGG CAT) were injected at the 1-4-cell stage into the yolk of zebrafish embryos according to published methods (Nasevicius and Ekker, 2000). The PMO was obtained from AVI Biopharma (Corvallis, Oreg.) and was injected at 3, 6 and 9 ng per embryo, we used 50 embroys per concentration. The experiment was repeated five times. Heart function and morphological anaylsis of larvae were scored one to 5 d later. Controls included the injection of only the injection buffer.

Of embryos that received 6 ng of the PMO, 67% showed the 2:1 heart beat at 2 days post-fertilization, and 28% showed an inrregular arrhythmia, the atrial beat rate strongly reduced and uncoordinated. All larvae showed a slight bradycardia and no other visible abnormalities. At day 3 the number of larvae with a 2:1 heart beat decreased to 10%, at day 5 all larvae were indistinguishable from wildtype larvae. This effect is probably due to the dilution of the PMOs in the larvae during development. There were slight variations in the percentage of larvae displaying the 2:1 phenotype between different experiments using 6 ng PMO, ranging between 60% and 100%. In the case of lower percentages, the phenotype was a little bit stronger, the rest of the embryos displaying irrregular arrhythmias. Increasing the amount of injected PMOs to 9 ng resulted in a stronger phenotype compared to 6 ng, 90% of the larvae had a strongly reduced heart beat rate and contractility, both chambers beating 1:1, and the circulation was strongly reduced or absent. Larvae showed a heart edema and blood accumulated on the yolk sac (day 2). Beginning with day 4 larvae developed a strong necrosis and died around day 6. Interestingly, the heart morphology of these larvae changed to a pattern known for dilative cardiomyopathy, chamber walls were very thin and chamber diameter enlarged. As expected, the phenotypes were less severe from injectioned of 3 ng of the PMO. While approximately 18% of larvae showed the 2:1 phenotype, approximately 81% showed a slight bradycardia, and approximately 5% appeared wild-type.

### Example 4

### ZERG Expression Analysis Using Whole-mount in Situ Histochemistry

Digoxigenin-labelled antisense and sense probes (3 kbp ZERG 3′ race product) were prepared according to the Boehringer instructions (Cat. No. 1175 025). Embryos were incubated in phenylthiourea (200 μM) to inhibit pigmentation. In brief, embryos were fixed with 4% paraformaldehyde, partially digested with proteinase K, and hybridized with the ZERG probe at 55° C. Alkaline-phosphatase-conjugated anti-digoxigenin (Boehringer Mannheim) was used to detect ZERG signals. After staining with BM purple (Boehringer Mannheim), embroys were washed with PBS and stored in 87% glycerol.

The presence of ZERG mRNA was detected at 24 hours post fertilization through 5 days post fertilization by whole-mount in situ hybridization revealing a strong and specific staining of the atrium and ventricle of wildtype and breakdance larvae. Thus, the expression pattern is similar to the expression in adult mammals.

In older larvae (21 days post-fertilization) a strong staining was seen in a particular region of the brain. Two narrow stripes of stained cells were detected in the tectum. Staining began close to the eyes and extended towards midbrain.

References

Abbott G W et al. Cell 97 (1999), 175-187.

Altschul et al., J. Mol. Biol. (1997) 215:403-410.

Ausubel F M et al. Current Protocols in Molecular Biology, John Wiley & Sons, New York, N.Y., 1994.

Babij P et al., Circ Res: 83:668-678 (1998).

Bateman A., et al., Nucleic Acids Res, 1999, 27:260-2.

Bauer C K et al., Receptors Channels 1998, 6:19-29.

Bianchi L et al., Cancer Res 1998, 58:815-822.

Capecchi, Science 1989, 244:1288-1292.

Chen et al., Development 123:293-302 (1996).

Cherubini A et al., Br J Cancer Research 2000, 83:1722-1729.

Coligan J E et al, Current Protocols in Protein Science (eds.), 1999, John Wiley & Sons, New York.

Crumb W and Cavero I, Pharm Sci Technol Today 2, 270-280 (1999).

Culp P et al., Proc Natl Acad Sci USA 1991, 88:7953-7957.

Demolombe S et al., Cardiovasc. Res. 50(2):314-327 (2001).

Draper et al., 2001, Genesis 30:154-6.

Drolet B, J. Pharmacol. Exp. Ther. 288, 1261-1268 (1999).

Eder C, Am J Physiol 1998, 275:C327-342.

Elbashir S M et al., Nature 2000, 411:494-498.

Emmi A et al., J Neurosci 2000, 20:3915-3925.

Francès V and Bastin M, Nucleic Acids Res 1996, 24:1999-2004.

Franz et al. Cardioscience 1994. 5:235-43.

Geelen P et al., Circulation 102, :275-277 (2000).

Gintant G A et al., J. Cardiovasc Pharmacol 2001, 37:607-618.

Higgins S J and Hames B D (eds.) Protein Expression: A Practical Approach, Oxford University Press Inc., New York 1999.

Hong et al., 1998, Proc Natl Acad Sci U S A 95:3679-3684

Hsiao C et al., Dev Biol 1999, 21:207-216.

Hunkapiller et al., Nature 1984, 310:105-111.

Huynen M A and Bork P, Proc Natl Acad Sci 1998, 95:5849-5856.

Huynen M A et al., Genome Research 2000, 10:1204-1210.

Itoh T. et al., Hum. Genet. 102, 435-439 (1998).

January C. T et al., Cardiovasc. Electrophysiol. 11, 1413-1418 (2000).

Joyner et al., Nature 1989, 338:153-156.

Ju et al., Dev Genet 1999, 25:158-67.

Kang J et al., Eur. J. Pharmacol. 31, 392, 137-140 (2000).

Kawakami K et al., Proc Natl Acad Sci U S A 2000, 97:11403-11408.

Koster R W and Fraser S E, Dev Biol 200, 233:329-346.

Laurita K R and Singal A, Am J Physiol Heart Circ Physiol 2001, 280:H2053-2060.

Lin S, Methods Mol Biol 2000, 136:375-383.

Linney E, Dev Dyn 2001, 220:323-336.

Liu J P et al, Cell 1993, 75:59-72.

Ma C et al., Proc Natl Acad Sci USA 2001, 98:2461-2466.

Mateyak M K et al., Cell Growth Differ 1997, 8:1039-1048.

Mitcheson J S et al., Proc. Natl. Acad. Sci. U S A 97, 12329-1233 (2000).

Mounsey J P and DiMarco J P, Circulation 2000, 102:2665-2670.

Muldoon R R et al., Biotechniques 1997, 22:162-167.

Nakai K and Horton, Trends Biochem Sci, 1999, 24:34-36.

Nakai K, Adv Protein Chem 2000, 54:277-344.

Nasevicius A and Ekker S C, Nat. Genet. 26, 216-220 (2000).

Numaguchi H et al., Circ. Res. 87, 1012-1018 (2000).

Ozato et al., 1986, Cell Differ 19:237-244.

Peterson R T et al., Proc Natl Acad Sci USA 2000, 97:12965-12969.

Prentice et al. Cardiovasc Res 1997, :567-74

Probst J C, Methods 2000, 22:271-281.

Rampe D et al., FEBS Lett 417:28-32 (1997).

Rampe D et al., Pharmacol. Exp. The. 286, 788-793 (1998).

Reaume A G et al., J Biol Chem 1996, 271:23380-23388.

Rong Y S and Golic K G, Science 2000, 288:2013-2018.

Rosati B et al., FASEB J 2000, 14:2601-2610.

Rothman et al., Gene Ther 1996, 3:919-26.

Saganich M J et al., J Neurosci 2001, 21:4609-4624.

Sambrook et al., Molecular Cloning 1989, Cold Spring Harbor Laboratory Press, New York

Sanguinetti M C et al., Cell 81, 299-307 (1995).

Sansom M S, Curr Biol 1999, 9:R173-R175.

Schmajuk et al., 1999, J Biol Chem 274:21783-9.

Schwerte T and Pelster B, J Exp Biol 2000, 203:1659-1669.

Soomets et al., 1999, Front Biosci 1999, 4:D782-6.

Splawski I. et al., Circulation 102, 1178-1185 (2000).

Suessbrich H et al., Br. J. Pharmacol. 120, 968-974 (1997)

Suessbrich H et al., FEBS Lett 385, 77-80 (1996).

Summerton J and Weller D, Antisense Nucleic Acid Drug Dev 1997, 7:187-95.

Taglialatela M et al., Clin. Exp. All. 29, Suppl. 3, 182-189 (1999).

Taglialatela M et al., Trends Pharmacol Sci 21, 52-56 (2000).

Takagi et al., 1994, Mol. Marine Biol. Biotech. 3:192-199.

Tanaka et al, 2001, Proc Natl Acad Sci U S A 98:2544-2549.

Thompson J D et al, 1994, Nucleic Acids Res 22:4673-4680.

Tinel N et al., FEBS Lett 2000, 480:137-141.

Tseng G N, J Mol Cell Cardiol 2001, 33:835-849.

Urtishak et al., 5th International Conference on Zebrafish Development and Genetics, Jun. 12-16 2002. Madison, Wis. Abstract 342, p. 17.

Vatta M et al., Curr. Opin. Cardiol. 15, 12-22 (2000).

Wakamatsu et al., 2001, Proc Natl Acad Sci U S A 98:1071-1076.

Westerfield M, The zebrafish book, University of Oregon Press, Eugene, Oreg., USA, 1993.

Wittbrodt et al. Nat Rev Genet 2002, 3:53-64.

Yamamoto K et al., Toxicol Sciences 2001, 60:165-176.

Yap Y. G. and Camm A. J Clin. Exp. All. 29, Suppl. 3, 174-181 (1999).

Yoshida H et al., Am J Med Genet 2001, 98:348-352.

Zhang W et al., Genes Dev 1995, 9:1388-1399.

It is claimed:

1. An isolated nucleic acid molecule comprising a polynucleotide sequence that encodes or is complementary to a sequence that encodes a teleost ERG polypeptide.

2. The nucleic acid molecule of claim 1 that hybridizes under high stringency conditions to the nucleic acid molecule having the polynucleotide sequence presented as SEQ ID NO:1, or the complement thereof.

3. The nucleic acid molecule of claim 1 wherein the nucleic acid molecule encodes the ZERG polypeptide having the amino acid sequence presented as SEQ ID NO:2.

4. The nucleic acid molecule of claim 1 comprising the polynucleotide sequence presented as SEQ ID NO:1, or the complement thereof.

5. An antisense oligomer capable of inactivating a ZERG gene comprising a nucleotide sequence complementary to at least 10 contiguous nucleotides within nucleotides 1-150 of SEQ ID NO:1.

6. An antisense oligomer of claim 5 having a nucleotide sequence complementary to 20-30 contiguous nucleotides within nucleotides 1-130 of SEQ ID NO:1.

7. An antisense oligomer of claim 5 that has the nucleotide sequence presented as SEQ ID NO:3.

8. An antisense oligomer of claim 5 that is a PMO.

9. An antisense oligomer of claim 7 that is a PMO.

10. A genetically modified zebrafish comprising an endogenous ZERG gene, wherein expression of the ZERG gene has been specifically disrupted by administration of an antisense oligomer of claim 5.

11. A genetically modified teleost comprising an endogenous teleost ERG gene wherein expression of the teleost ERG gene has been specifically disrupted by admininstration of a PMO comprising a nucleotide sequence complementary to a fragment of the teleost ERG gene.

12. A method of identifying a cardio-active agent comprising the steps of:
a) providing mutant teleost larvae having reduced teleost ERG activity;
b) contacting the teleost larvae with a candidate agent;
c) detecting an agent-biased cardiac phenotype in the mutant teleost larvae,
wherein detection of an agent-biased cardiac phenotype indicates that the candidate agent is a cardio-active agent.

13. The method of claim 12 wherein the cardiac phenotype is chosen from the group consisting of irregular arrhythmia, bradycardia, 2:1 arrhythmia, rescue of 2:1 arrhythmia, aberrant heart morphology, lack of circulation, and blood accumulation in the yolk.

14. The method of claim 12 wherein the cardiac phenotype is detected using visual detection methods.

15. The method of claim 12 wherein the candidate agent is a small molecule compound.

16. The method of claim 12 wherein the cardio-active agent is an anti-arrhythmic agent.

17. The method of claim 12 wherein the cardio-active agent is a pro-arrhythmic agent.

18. The method of claim 12 wherein the mutant teleost larvae having reduced teleost ERG activity are zebrafish larvae.

19. The method of claim 18 wherein the zebrafish larvae are breakdance larvae.

20. The method of claim 18 wherein the zebrafish larvae are wild-type larvae treated with ZERG-specific PMOs.

21. The method of claim 20 wherein the ZERG-specific PMOs have the nucleotide sequence presented as SEQ ID NO:3.

22. The method of claim 12 wherein the mutant teleost larvae having reduced teleost ERG activity express the HERG gene.

23. A chimeric ZERG gene encoding a chimeric polypeptide have an amino acid sequence derived from the sequence presented as SEQ ID NO:2, wherein the chimeric polypeptide comprises at least one sequence replacement selected from the group consisting of:
(i) replacing amino acid 376 of SEQ ID NO:2 with amino acid 413 of SEQ ID NO:5,
(ii) replacing amino acids 388-413 of SEQ ID NO:2 with amino acids 425-451 of SEQ ID NO:5,
(iii) replacing amino acids 414-434 of SEQ ID NO:2 with amino acids 452-472 of SEQ ID NO:5,
(iv) replacing amino acids 435-458 of SEQ ID NO:2 with amino acids 437-496 of SEQ ID NO:5,
(v) replacing amino acid 476 of SEQ ID NO:2 with amino acid 514 of SEQ ID NO:5,
(vi) replacing amino acids 540-575 of SEQ ID NO:2 with amino acids 573-603 of SEQ ID NO:5,
(vii) replacing amino acid 608 of SEQ ID NO:2 with amino acid 636 of SEQ ID NO:5.

24. A chimeric ZERG gene of claim 23, wherein the chimeric ZERG gene encodes a chimeric polypeptide comprising the sequence presented as SEQ ID NO:4.

25. A transgenic zebrafish comprising a chimeric gene of claim 23.
